(19) United States
(12) Patent Application Publication
(10) Pub. No.: US 2022/0361740 A1
(43) Pub. Date: Nov. 17, 2022

(54) METHODS AND APPARATUS FOR A CLEANING SHEATH FOR AN ENDOSCOPE

(71) Applicant: Hao Kenith Fang, Scottsdale, AZ (US)

(72) Inventor: Hao Kenith Fang, Scottsdale, AZ (US)

(21) Appl. No.: 17/875,164

(22) Filed: Jul. 27, 2022

Related U.S. Application Data
(63) Continuation-in-part of application No. 16/401,319, filed on May 2, 2019.
(60) Provisional application No. 62/670,205, filed on May 11, 2018.

(51) Int. Cl. A61B 1/12; A61B 1/00

(52) U.S. Cl. CPC A61B 1/126; A61B 1/00135; A61B 1/00128

(57) ABSTRACT

A cleaning sheath for an endoscope includes an open ended sheath coupled to a fluid line via a hub. The sheath is configured to receive an endoscope within the interior of the sheath wherein both the sheath and the endoscope may be inserted into a trocar during use. The sheath is configured to direct a cleaning fluid onto the optics end of the endoscope to clear away any visual obstructions without requiring the removal of the endoscope from the trocar. The hub is configured to hold the endoscope in a desired position during use to allow a user to selectively adjust and fix the position of the optics end of the endoscope relative to the distal end of the sheath during use at a location extending beyond the distal end of the sheath, aligned with the distal end of the sheath, or withdrawn into the distal end of the sheath.

### CROSS-REFERENCES TO RELATED APPLICATIONS

This application is a continuation-in-part application of U.S. Nonprovisional patent application Ser. No. 16/401,319, filed on May 2, 2019 which claims the benefit of U.S. Provisional Patent Application No. 62/670,205, filed May 11, 2018, and incorporates the disclosure of the application by reference.

### BACKGROUND OF THE TECHNOLOGY

Endoscopes are used in a variety of surgical procedures including endoscopy, laparoscopy, and thoracoscopy to investigate symptoms in the digestive system, cardiovascular system, and the sinuses. A common frustration experienced in procedures is the altered and obstructed visualization caused by debris and bodily fluids that come into contact with the distal fiberoptic tip of the endoscope. In addition, upon first insertion of the endoscope into a warm body cavity, the lens of the optics tip may fog similar to eye goggles when swimming or snorkeling. When this occurs, it typically requires the removal of the endoscope from the body cavity to remove the obstructing substance manually. Removing and reinserting the endoscope wastes valuable operating room time and is potentially dangerous when visualization is compromised during a critical point of an operation/procedure.

Existing solutions directed towards maintaining a clear field of vision after the tip of the endoscope has been inserted into a body cavity include devices that do not function with existing sizes of associated medical devices used during the surgical procedure such as a trocar. Due to the variable lengths of endoscopes used within the field, existing solutions are not able to perform universally with a given diameter endoscope. Therefore, existing devices that are used to clean the optics end of an endoscope are typically limited to being used with a particular endoscope having a particular diameter and a specific length. If the length of a second endoscope does not have the same exact length it cannot be used thereby limiting the usefulness of the cleaning device in a real-world surgical environment.

Existing devices often also require separate pumps, motors, electronics, and switches to function properly. Each of these devices increases the number of equipment located where the surgical procedure is being performed taking up space and increasing costs. These device are often bulkier and more cumbersome which increases the workload on surgical staff. Further, introducing additional components during the surgical procedure results in increased complexity and requires additional training by medical staff.

### SUMMARY OF THE TECHNOLOGY

A cleaning sheath for an endoscope includes an open ended sheath coupled to a fluid line via a hub. The sheath is configured to receive an endoscope within the interior of the sheath wherein both the sheath and the endoscope may be inserted into a trocar during use. The sheath is configured to direct a cleaning fluid onto the optics end of the endoscope to clear away any visual obstructions without requiring the removal of the endoscope from the trocar. The hub is configured to hold the endoscope in a desired position during use to allow a user to selectively adjust and fix the position of the optics end of the endoscope relative to the distal end of the sheath during use at a location extending beyond the distal end of the sheath, aligned with the distal end of the sheath, or withdrawn into the distal end of the sheath.

### BRIEF DESCRIPTION OF THE DRAWINGS

A more complete understanding of the present technology may be derived by referring to the detailed description and claims when considered in connection with the following illustrative figures. In the following figures, like reference numbers refer to similar elements and steps throughout the figures.

FIG. 1 representatively illustrates a side view of an endoscope sheath in accordance with an exemplary embodiment of the present technology;

FIG. 2 representatively illustrates a top view of the endoscope sheath shown in FIG. 1 in accordance with an exemplary embodiment of the present technology

FIG. 3 representatively illustrates the endoscope sheath coupled to a syringe with the endoscope partially inserted into the endoscope sheath in accordance with an exemplary embodiment of the present technology;

FIG. 4 representatively illustrates the endoscope sheath coupled to a syringe inserted into the endoscope sheath so that the distal end of the sheath and optics end of the endoscope are aligned in accordance with an exemplary embodiment of the present technology;

FIG. 5 representatively illustrates the endoscope sheath coupled to a syringe with the endoscope fully inserted into the endoscope sheath so that the optics end of the endoscope extends beyond the sheath in accordance with an exemplary embodiment of the present technology;

FIG. 6 representatively illustrates a cross-sectional view across line 6-6 of a fluid hub shown in FIG. 2 in accordance with an exemplary embodiment of the present technology;

FIG. 7 representatively illustrates a perspective view of a valve seal in accordance with an exemplary embodiment of the present technology;

FIG. 8 representatively illustrates a detailed cross-sectional view of the valve seal positioned within the fluid hub in accordance with an exemplary embodiment of the present technology;

FIG. 9 representatively illustrates an optics end of an endoscope being inserted into the endoscope sheath in accordance with an exemplary embodiment of the present technology;

FIG. 10 representatively illustrates the optics end of the endoscope inserted part way through the endoscope sheath in accordance with an exemplary embodiment of the present technology;

FIG. 11 representatively illustrates the optics end of the endoscope being inserted fully into the endoscope sheath in accordance with an exemplary embodiment of the present technology;

FIG. 12 representatively illustrates the optics end of the endoscope and the endoscope sheath being inserted into a trocar in accordance with an exemplary embodiment of the present technology;

FIG. 13 representatively illustrates the optics end of the endoscope and the endoscope sheath being fully inserted into the trocar in accordance with an exemplary embodiment of the present technology;

FIG. 14 representatively illustrates a syringe being connected to a fluid line of the endoscope sheath in accordance with an exemplary embodiment of the present technology;

FIG. 15 representatively illustrates a display of the optics end of the endoscope in accordance with an exemplary embodiment of the present technology;

FIG. 16 representatively illustrates the display of the optics end of the endoscope with a visual obstruction in accordance with an exemplary embodiment of the present technology;

FIG. 17 representatively illustrates the display of the optics end of the endoscope at the beginning of a flush cycle in accordance with an exemplary embodiment of the present technology;

FIG. 18 representatively illustrates the display of the optics end of the endoscope during the flush cycle in accordance with an exemplary embodiment of the present technology; and

FIG. 19 representatively illustrates the display of the optics end of the endoscope at the completion of the flush cycle in accordance with an exemplary embodiment of the present technology.

Elements and steps in the figures are illustrated for simplicity and clarity and have not necessarily been rendered according to any particular sequence. For example, steps that may be performed concurrently or in a different order are illustrated in the figures to help to improve understanding of embodiments of the present technology.

### DETAILED DESCRIPTION OF EXEMPLARY EMBODIMENTS

The present technology may be described in terms of functional block components and various processing steps. Such functional blocks may be realized by any number of components configured to perform the specified functions and achieve the various results. For example, the present technology may employ various materials, coupling mechanisms, dimensions, and geometries, which may carry out a variety of operations suited to a selective attachment to or use with an endoscope, trocar, or syringe. In addition, the technology described is merely one exemplary application for the invention. Further, the present technology may employ any number of conventional techniques for flushing, cleaning, or otherwise clearing debris such as bodily fluids and fog from optical devices.

Methods and apparatus for an endoscope sheath according to various aspects of the present technology may operate in conjunction with any type of endoscope, fiberoptic video capture system, or micro-camera. Various representative implementations of the present technology may be applied to any type of viewing device that is insertable or otherwise intended for use within a body during a medical procedure that may be subjected to various types of visual obstructions.

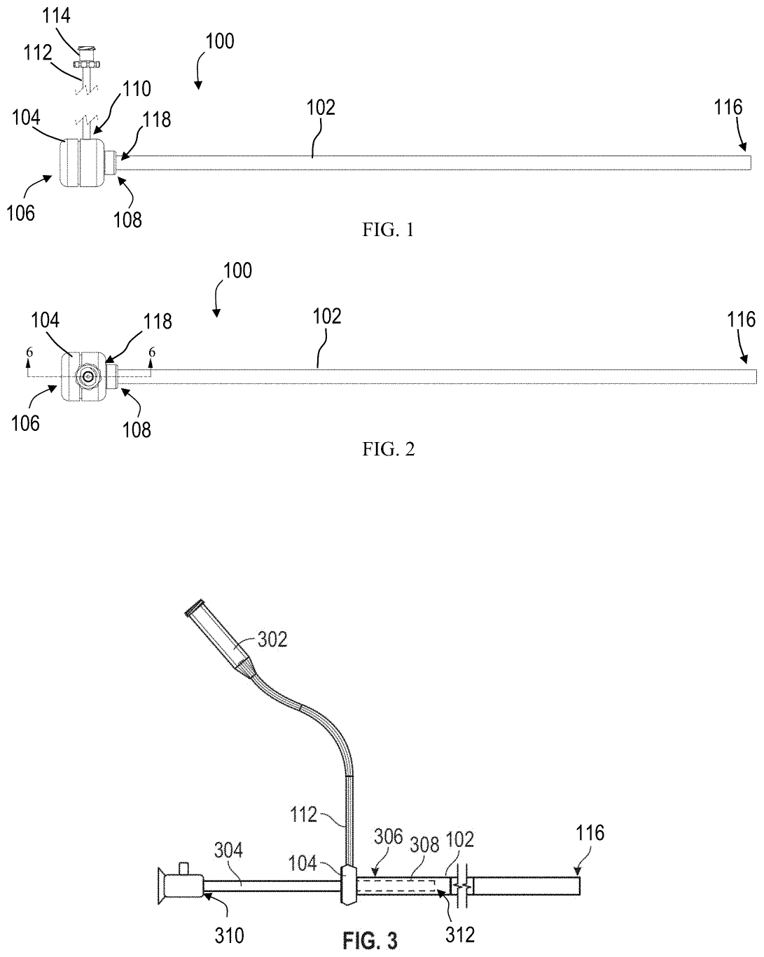

Referring to FIGS. 1-3, a cleaning sheath 100 may comprise a tubular shaped body 102 having a distal end 116 and a proximal end 118. An interior of the body 102 is configured to receive a viewing device such as an endoscope 304. The interior of the body 102 may form a lumen extending between the proximal and distal ends 118, 116 to both receive the endoscope and allow a flow of cleaning fluid, such as a saline solution, from the proximal end 118 to the distal end 116. A fluid hub 104 may be coupled to the proximal end 118 of the body 102 and be in fluid communication with the lumen. A fluid line 112 may be coupled to the fluid hub 104 and be configured to provide a conduit path for the cleaning fluid.

The body 102 may comprise any suitable size or shape and may be selected according to any applicable criteria such as a diameter of the viewing device that the body 102 will be positioned over. For example, the body 102 may comprise a single walled sheath having an external diameter of between 2 mm and 14 mm to allow both the endoscope 304 and the body 102 to be positioned within a trocar (See FIG. 12) as commonly used in medical procedures. An interior surface of the sheath may comprise a smooth continuous surface between the proximal and distal ends 116, 118 that is free from protrusions, ridges, channels, or other elements that may contact, obstruct, or impede the movement of the endoscope 304 through the body 102.

An internal diameter of the body 102 may be sized slightly larger than the outer diameter of the endoscope 304 to allow the cleaning fluid to flow completely around an outer surface 308 of the endoscope towards the distal end 116 of the body 102. For example, and in one embodiment where the endoscope 304 comprises a 5 mm thoracoscope having an outer working diameter of about 5.4 mm, the body 102 may comprise an inner diameter of between 5.42 mm and 5.75 mm. The larger diameter of the body 102 relative to the endoscope 304 creates a fluid conduit 306 that extends completely around the outer surface 308 of the endoscope and along the length of the body 102 between the proximal and distal ends 118, 116 in which the cleaning fluid may flow.

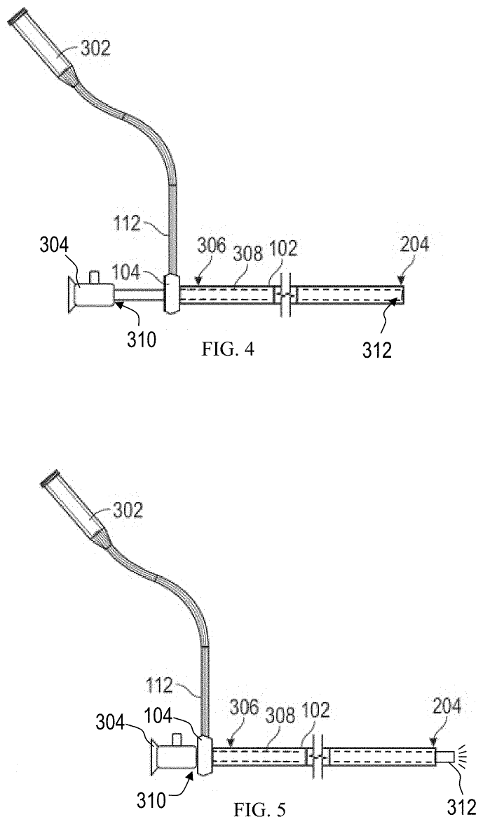

The body 102 comprises a length such that the distal end 116 extends no further than an optics 312 end of the endoscope 304 when the endoscope is fully inserted into the cleaning sheath 100 and a base 310 of the endoscope is positioned directly against the fluid hub 104. Referring now to FIGS. 4 and 5, the body 102 may comprise a length less than that of the endoscope such that the optics end 312 of the endoscope may extend beyond the distal end 116 of the body 102 if the endoscope is fully inserted. In use, this means that the optics end 312 of the endoscope 304 may be aligned with the distal end 116 of the body without requiring the endoscope to be fully inserted (See FIG. 4). The optics end 312 may also be positioned beyond the distal end 116 of the body 102 during use and only retracted back into body 102 if a visual obstruction needs to be cleaned off of the optics end 312 (See FIG. 5). One of skill in the art will appreciate that the cleaning sheath 100 may be used with varying lengths of endoscopes having a diameter that corresponds to that of the body 102 rather than being limited to a specific endoscope of a set diameter and set length.

The distal end 116 of the body 102 is configured without any obstructions or other elements that may engage, contact, or otherwise prevent the optics end 312 of the endoscope 304 from extending through the distal end 116 of the body 102. More specifically, the distal end 116 is configured to allow the optics end 312 to freely extend past a terminal edge of the distal end 116 of the body 102 without coming into contact with an interior surface of the body 102.

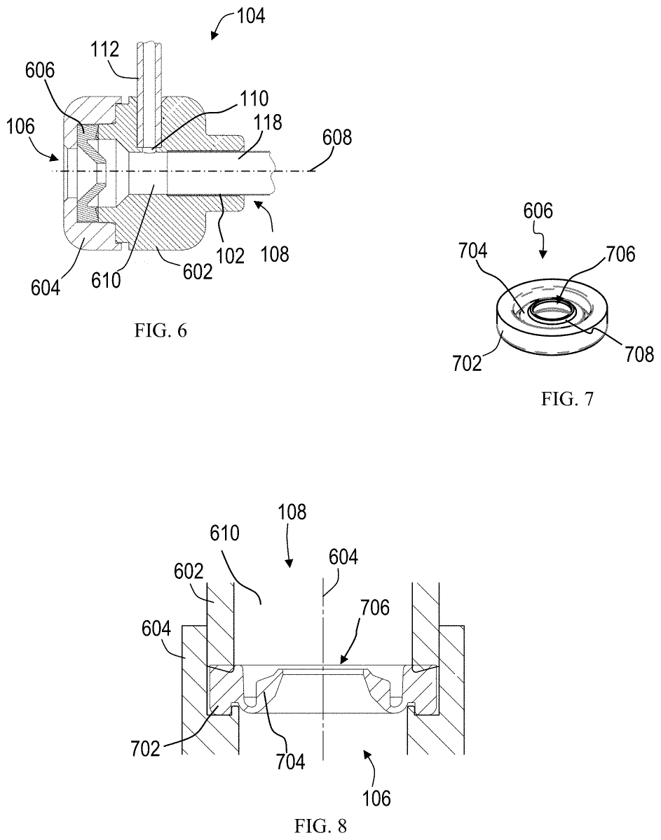

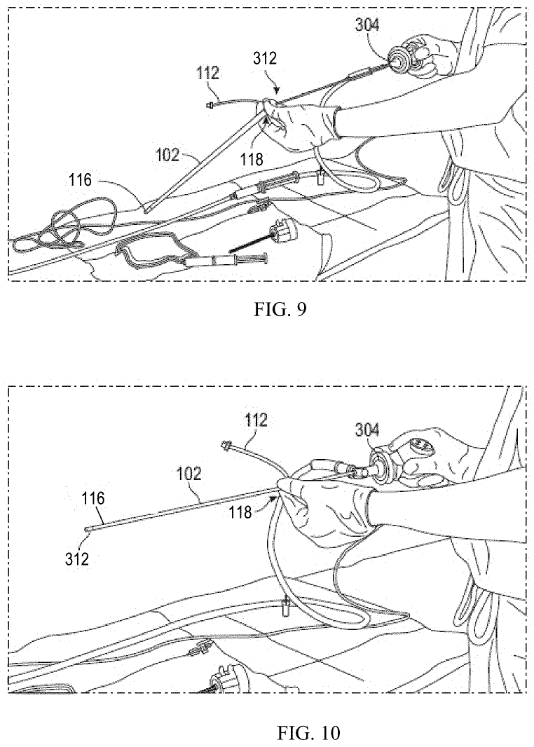

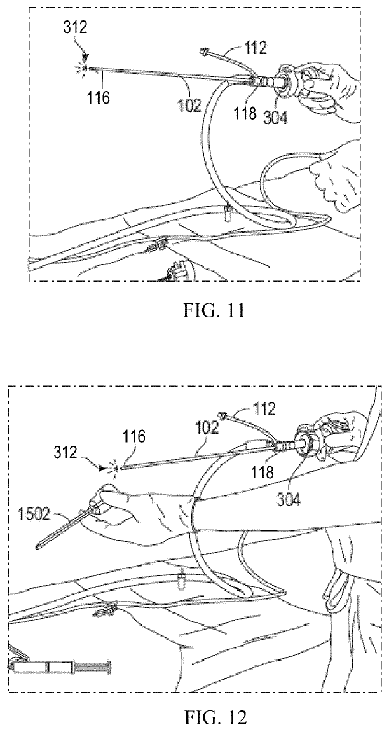

Referring now to FIGS. 1, 2, and 6-8 the fluid hub 104 provides a fluid link between the fluid line 112 and the lumen of the body 102. In one embodiment, the fluid hub 104 may comprise a main body 602 connected to a cap 604. A hub lumen 610 extends between opposing first and second open ends 106, 108 that form an insertion path for the endoscope 304 along a radial axis 608 of the lumen of the body 102. The second open end 108 of the fluid hub 104 receives and is coupled to the proximal end 118 of the body 102.

An inlet port 110 disposed between the first and second open ends 106, 108 is open to the hub lumen 610 and is coupled to the fluid line 112. The fluid line 112 may be coupled to the inlet port 110 by any suitable method. In one embodiment, the inlet port 110 may be configured with a compression fitting configured to create a seal between the fluid line 112 and the interior of the fluid hub 104. In an alternative embodiment, the inlet port 110 may comprise an opening approximately equal to or slightly less than an outer diameter of a first end of the fluid line 112 with an edge configured to create a seal against the fluid line 112 when an end of the fluid line 112 is inserted through the opening.

The cap 604 of the fluid hub 104 may be configured to hold the endoscope 304 in position along the radial axis 608 of the body 102 and create a seal against the endoscope 304 when it is inserted through the fluid hub 104 to prevent fluid from exiting out of the first open end 106 during use. For example, the cap 604 may comprise a valve seal 606 positioned between an outer edge of the first open end 106 and the inlet port 110. In one embodiment, the valve seal 606 may comprise a rigid disc 702 having a flexible inner seal 704 disposed along an inner portion of the rigid disc 702. The flexible inner seal 704 may comprise an opening 706 having a center aligned with the radial axis 608. The opening 706 may comprise an inner diameter approximately equal to or slightly less than the of the diameter of the outer surface 308 of the endoscope 304 that is intended to be used with the cleaning sheath 100.

The flexible inner seal 704 may be configured to flex and allow the opening 706 to expand slightly to accommodate the endoscope 304. A lip 708 of the inner seal 704 may be configured to create a seal against the outer surface 308 of the endoscope 304 to prevent fluid from egressing out of the first open end 106 when fluid is flowing into the hub lumen 610.

The lip 708 may further be configured to grip the outer surface 308 of the endoscope 304 with a sufficient force to hold the endoscope 304 in position such that it will not move freely along the radial axis 608. For example, the lip 708 may comprise a flat surface configured to engage the outer surface of the endoscope. A length or width of the flat surface may be selected to provide more or less surface area of contact between the lip 708 and the outer surface of the endoscope to adjust a surface tension level between the lip 708 and the endoscope. In a first embodiment, the lip may comprise a length of between about 0.75 mm and about 1.25 mm. In an alternative embodiment, the lip may be configured to provide an increased holding pressure as compared to the first embodiment and comprise a length of between about 1.1 mm and about 1.35 mm. The increase in surface area of the lip 708 will increase the area of contact between the lip 708 and the outer surface of the endoscope providing an increased holding pressure on the endoscope along the radial axis 608. This decreases the chances of the endoscope sliding or moving along the radial axis 608 of the lumen of the body 102 unintentionally during use. This allows the user to position the endoscope in a desired location along the body 102 and have it held in place.

Referring again to FIGS. 1-3, the fluid line 112 may comprise any suitable system or device for creating a fluid conduit path between a cleaning fluid supply source and the fluid hub 104. For example, the fluid line 112 may comprise a flexible tube made from a polymer. The fluid line 112 may comprise any suitable length or diameter. For example, in one embodiment, the fluid line 112 may comprise a length of between about 50 mm and about 300 mm and comprise a diameter between about 2.5 mm and about 25 mm.

As described above, the first end of the fluid line 112 may be coupled to the inlet port 110 at or near the proximal end 202 of the body 102. A second end of the fluid line 112 may be configured to be connected to the cleaning fluid supply. For example, in one embodiment, the second end of the fluid line 112 comprise a leur lock 114 that may be selectively connected to an end of a syringe 302 containing the cleaning fluid.

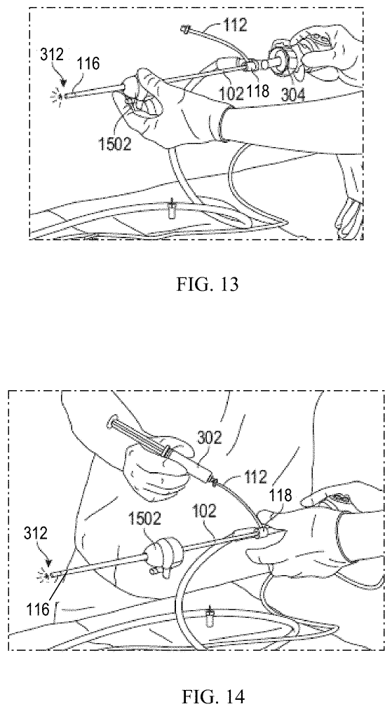

Referring now to FIGS. 12-22, in use the cleaning sheath 100 is used in conjunction with an endoscope 304 during the medical procedure and is configured to direct a cleaning fluid towards the optics end 312 of the endoscope 304 to clear away any visual obstruction. With particular reference to FIGS. 12-14, the optics end 312 may be inserted into the first end of the fluid hub 104 and pushed towards the distal end 204 of the body 102 until the optics end 312 is positioned at the terminal opening of the distal end 204.

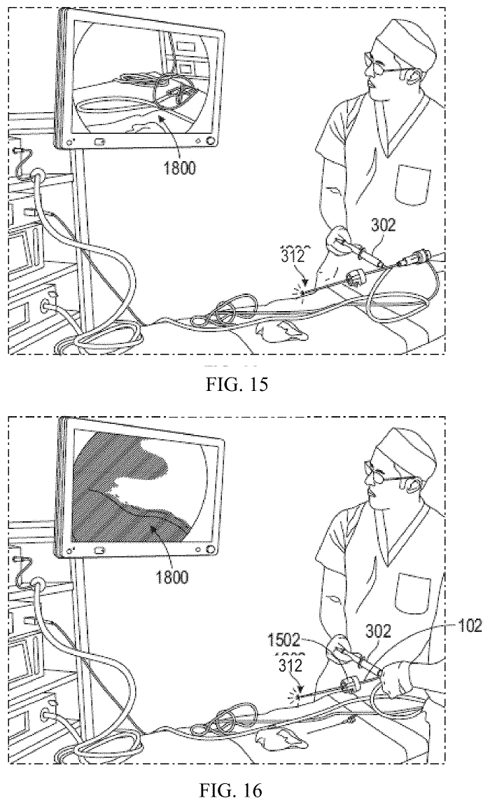

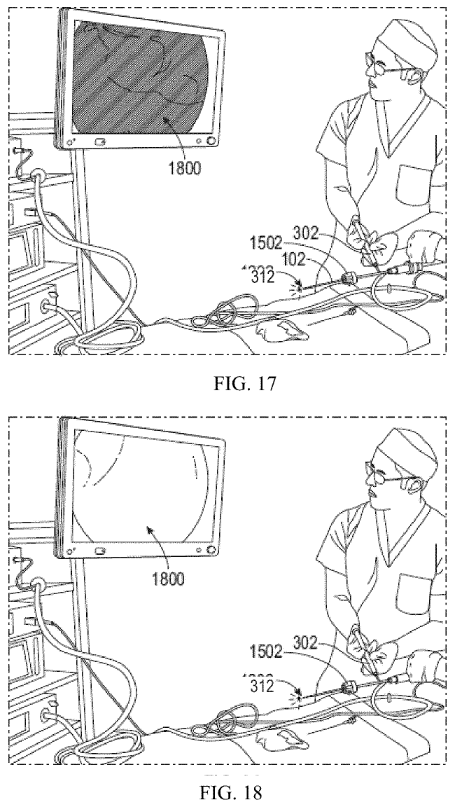

Referring now to FIGS. 15 and 16, during the medical procedure the combined endoscope 304 and the distal end 204 of the cleaning sheath 100 may be inserted through a trocar 1502 and subsequently into the body of the patient. As shown in FIG. 17, a syringe 302 containing the fluid supply may be connected to the second end of the fluid line 112.

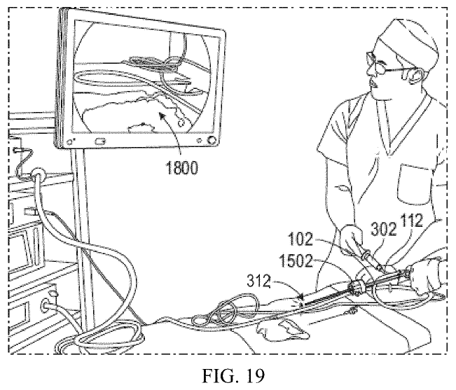

As shown in FIG. 18 the optics end 312 of the endoscope provides an image to a display device 1800 that may be viewed by the person performing the medical procedure. During the procedure, the optics end 312 may become obstructed by coming into contact with debris or a bodily fluid or due to a difference in temperature between the optics end 312 and the internal temperature of the body. This results in an obstructed field of view on the display device 1800 as shown in FIG. 19.

Referring now to FIGS. 19 and 20, when the field of view becomes obstructed, blurred, or otherwise blocked, cleaning fluid from the syringe 302 may be injected into the body 102. The cleaning fluid may flow through the fluid conduit 306 and out the distal end 204 of the body 102 and over the optics end 312 of the endoscope 304. After the cleaning fluid is passed over the optics end 312 the obstruction may be cleared away and the field of vision restored as shown in FIG. 22 without the need to remove the endoscope 304 from the body.

These and other embodiments for methods of creating a cleaning sheath may incorporate concepts, embodiments, and configurations as described above. The particular implementations shown and described are illustrative of the technology and its best mode and are not intended to otherwise limit the scope of the present technology in any way. Indeed, for the sake of brevity, conventional manufacturing, connection, preparation, and other functional aspects of the system may not be described in detail. Furthermore, the connecting lines shown in the various figures are intended to represent exemplary functional relationships and/or physical couplings between the various elements. Many alternative or additional functional relationships or physical connections may be present in a practical system.

The description and figures are to be regarded in an illustrative manner, rather than a restrictive one and all such modifications are intended to be included within the scope of the present technology. Accordingly, the scope of the technology should be determined by the generic embodiments described and their legal equivalents rather than by merely the specific examples described above. For example, the components and/or elements recited in any apparatus embodiment may be assembled or otherwise operationally configured in a variety of permutations to produce substantially the same result as the present technology and are accordingly not limited to the specific configuration recited in the specific examples.

As used herein, the terms “comprises,” “comprising,” or any variation thereof, are intended to reference a non-exclusive inclusion, such that a process, method, article, composition or apparatus that comprises a list of elements does not include only those elements recited but may also include other elements not expressly listed or inherent to such process, method, article, composition or apparatus. Other combinations and/or modifications of the above-described structures, arrangements, applications, proportions, elements, materials or components used in the practice of the present technology, in addition to those not specifically recited, may be varied or otherwise particularly adapted to specific environments, manufacturing specifications, design parameters or other operating requirements without departing from the general principles of the same. Any terms of degree such as “substantially,” “about,” and “approximate” as used herein mean a reasonable amount of deviation of the modified term such that the end result is not significantly changed. For example, these terms can be construed as including a deviation of at least ±5% of the modified term if this deviation would not negate the meaning of the word it modifies.

The present technology has been described above with reference to exemplary embodiments. However, changes and modifications may be made to the exemplary embodiments without departing from the scope of the present technology. These and other changes or modifications are intended to be included within the scope of the present technology, as expressed in the following claims.

1. A cleaning sheath for directing a fluid from a syringe to an optics portion of an endoscope, comprising:
a single walled sheath having a lumen extending between an open proximal end and an open distal end, wherein:
the sheath is configured to receive the endoscope within the proximal end and allow the optics portion to extend through the lumen and out of the distal end; and
a fluid conduit is formed around an outer surface of the endoscope and extending along the length of the single walled sheath between the open proximal end and the open distal end; and
a fluid hub, comprising:
opposing first and second open ends;
a hub lumen extending between the opposing first and second open ends, wherein:
the hub lumen forms an insertion path for the endoscope;
the proximal end of the sheath is positioned within and coupled to the second open end of the fluid hub; and
the hub lumen is in fluid communication with the fluid conduit;
an inlet port in fluid communication with the hub lumen; and
a valve seal disposed between the first open end and the inlet port, wherein the valve seal comprises:
a disc body; and
a flexible inner seal disposed along an inner portion of the disc body and comprising an opening having a center aligned with the hub lumen, wherein the flexible inner seal is configured to:
create a seal around the endoscope; and
hold the endoscope in place.

2. A cleaning sheath according to claim 1, wherein the fluid hub comprises:
a main body comprising the inlet port and the second open end; and
a cap coupled to the main body and configured to hold the valve seal.

3. A cleaning sheath according to claim 1, wherein the flexible inner seal comprises a lip configured to create a seal against an outer surface of the endoscope.

4. A cleaning sheath according to claim 3, wherein the seal lip comprises a flat surface configured to fit against the endoscope and create a surface tension between the lip and the outer surface of the endoscope to hold the endoscope in position relative to the lumen of the single walled sheath.

5. A cleaning sheath according to claim 1, wherein the fluid line comprises a flexible tube extending between the first and second ends.

6. A cleaning sheath according to claim 5, wherein the second end of the fluid line is configured to be removably coupled to the syringe.

7. A cleaning sheath according to claim 1, wherein the sheath is rigid between the proximal and distal ends.

8. A cleaning sheath according to claim 1, wherein the distal end of the sheath is angled between zero and ninety degrees.

9. A cleaning sheath for directing a fluid from a syringe to an optics portion of an endoscope, comprising:
a single walled sheath having a lumen extending between an open proximal end and an open distal end, wherein:
the sheath is configured to receive the endoscope within the proximal end and allow the optics portion to extend through the lumen and out of the distal end; and
a fluid conduit is formed around an outer surface of the endoscope and extending along the length of the single walled sheath between the open proximal end and the open distal end; and
a fluid hub, comprising:
opposing first and second open ends;
a hub lumen extending between the opposing first and second open ends, wherein:
the hub lumen forms an insertion path for the endoscope;
the proximal end of the sheath is positioned within and coupled to the second open end of the fluid hub; and
the hub lumen is in fluid communication with the fluid conduit;
an inlet port in fluid communication with the hub lumen; and
a valve seal disposed between the first open end and the inlet port, wherein the valve seal comprises:
a disc body; and
a flexible inner seal disposed along an inner portion of the disc body and comprising an opening having a center aligned with the hub lumen, wherein the flexible inner seal is configured to:
create a seal around the endoscope; and
hold the endoscope in place; and
a fluid line comprising:
a first end coupled to the inlet port; and
a second end configured to be removably coupled to the syringe.

10. A cleaning sheath according to claim 9, wherein the fluid hub comprises:
a main body comprising the inlet port and the second open end; and
a cap coupled to the main body and configured to hold the valve seal.

11. A cleaning sheath according to claim 9, wherein the flexible inner seal comprises a lip configured to create a seal against an outer surface of the endoscope.

12. A cleaning sheath according to claim 11, wherein the seal lip comprises a flat surface configured to fit against the endoscope and create a surface tension between the lip and the outer surface of the endoscope to hold the endoscope in position relative to the lumen of the single walled sheath.

13. A cleaning sheath according to claim 9, wherein the distal end of the sheath is angled between zero and ninety degrees.
